(19) United States
(12) Patent Application Publication
(10) Pub. No.: US 2015/0065878 A1
(43) Pub. Date: Mar. 5, 2015

(54) METHODS AND APPARATUSES FOR MONITORING TEMPERATURE CHANGE OF REGION OF INTEREST BY USING PERIODIC BIO-SIGNALS OF OBJECT

(71) Applicant: SAMSUNG ELECTRONICS CO., LTD., Suwon-si (KR)

(72) Inventors: Joon-ho SEO, Yongin-si (KR); Sun-kwon KIM, Yongin-si (KR); Won-chul BANG, Yongin-si (KR); Jong-bum SON, Yongin-si (KR)

(73) Assignee: SAMSUNG ELECTRONICS CO., LTD., Suwon-si (KR)

(21) Appl. No.: 14/476,079

(22) Filed: Sep. 3, 2014

(30) Foreign Application Priority Data
Sep. 3, 2013 (KR) 10-2013-0105694

(51) Int. Cl. A61B 8/08; A61B 8/00; A61B 5/08; A61B 8/14; A61B 5/024

(52) U.S. Cl. CPC A61B 8/5269; A61B 8/14; A61B 8/5223; A61B 5/024; A61B 5/0816; A61B 8/469
USPC 600/438

(57) ABSTRACT

A method of generating an image by using ultrasound waves. The method includes acquiring a first bio-signal of an object, generating at least one first ultrasound frame by using at least one echo signal obtained from at least one first ultrasound wave transmitted to a preset region of interest (ROI) of the object, generating a second ultrasound frame by using an echo signal obtained from a second ultrasound wave transmitted to the preset ROI, and acquiring a second bio-signal of the object at a time when the second ultrasound wave is transmitted to be used for generating the second ultrasound frame, and determining a reference frame from among the generated at least one first ultrasound frame based on the generated second ultrasound frame and the acquired second bio-signal.

### CROSS-REFERENCE TO RELATED APPLICATIONS

This application claims the benefit of Korean Patent Application No. 10-2013-0105694, filed on Sep. 3, 2013, in the Korean Intellectual Property Office, the disclosure of which is incorporated herein in its entirety by reference.

### BACKGROUND

1. Field

One or more exemplary embodiments relates to methods and apparatuses for monitoring a temperature of a region of interest (ROI) by using periodic bio-signals of an object.

2. Description of the Related Art

An ultrasound image of a preset ROI of an object is generated by transmitting an ultrasound wave to the object and using an echo signal reflected from the object. The ultrasound image of the ROI includes a temperature image indicating a temperature of a cross-sectional surface of the ROI, or a brightness (B)-mode image indicating the brightness of the cross-sectional surface of the ROI. A traveling speed of the ultrasound wave for generating an ultrasound image changes according to a temperature of the object.

In monitoring a temperature change of an ROI of an object by using an echo signal, a temperature of the ROI cannot be accurately monitored due to a change in a bio-signal of the object. For example, a tissue included in the ROI may translate or rotate and a shape of the tissue may deform due to changes in the bio-signal of the object.

### SUMMARY

Provided are methods and apparatuses for monitoring a temperature of an ROI by using periodic bio-signals of an object.

Provided is a non-transitory computer-readable storage medium storing a program for executing the method in a computer.

Additional aspects will be set forth in part in the description which follows and, in part, will be apparent from the description, or may be learned by practice of exemplary embodiments.

According to an aspect of one or more exemplary embodiment, a method of generating an image by using ultrasound waves includes: acquiring a first bio-signal of an object; generating at least one first ultrasound frame by using at least one echo signal obtained from at least one first ultrasound wave transmitted to a preset region of interest (ROI) of the object; generating a second ultrasound frame by using an echo signal obtained from a second ultrasound wave transmitted to the preset ROI, and acquiring a second bio-signal of the object at a time when the second ultrasound wave is transmitted; and determining a reference frame from among the generated at least one first ultrasound frames on a basis of the generated ultrasound frame and the acquired bio-signal.

The method may further include acquiring information about a temperature change of the preset ROI by using the generated second ultrasound frame and the determined reference frame.

The method may further include generating a model based on a period of the first bio-signal by using the acquired first bio-signal and the generated at least one first ultrasound frames, wherein the determining of the reference frame comprises determining the reference frame from the model based on the generated second ultrasound frame and the acquired second bio-signal.

The model may be based on grouping the at least one first ultrasound frame based on time intervals included in the period of the first bio-signal.

The generating of the model may include separating the acquired first bio-signal into units of one period; combining the separated one period units of the first bio-signal to generate a reference bio-signal; and generating the model by mapping the at least one first ultrasound frame to the reference bio-signal based on time intervals included in the reference bio-signal.

The separating may include detecting a time when the first bio-signal repeats similar patterns and determining peak value of the bio-signal, by using the acquired bio-signal; and separating the bio-signal into units of one period by using the detected time and the peak values.

The reference frame may be a frame that has a highest correlation with the generated second ultrasound from among the generated at least one first ultrasound frames.

The method may further include storing the generated at least one first ultrasound frames.

According to another aspect of one or more exemplary embodiment, provided is a non-transitory computer-readable storage medium storing a computer program for executing the method in a computer.

According to another aspect of one or more exemplary embodiment, an apparatus for generating an image by using ultrasound waves includes: a bio-signal acquirer configured to acquire a first bio-signal of an object; an ultrasound frame generator configured to generate a plurality of first ultrasound frames by using echo signals obtained from first ultrasound waves transmitted to a preset ROI of the object; and a reference frame determiner configured to determine a reference frame from among the plurality of first ultrasound frames on a basis of a second ultrasound frame and a second bio-signal, wherein, the ultrasound frame generator may be further configured to generate the second ultrasound frame by using an echo signal obtained from a second ultrasound wave which is transmitted to the ROI, and the bio-signal acquirer is further configured to acquire the second bio-signal at a time when the second ultrasound wave is transmitted to the preset ROI.

The apparatus may further include a temperature information acquirer configured to acquire information about a temperature change of the preset ROI by using the second ultrasound frame and the reference frame.

The apparatus may further include a model generator configured to generate a model based on a period of the first bio-signal by using the acquired first bio-signal and the generated first ultrasound frames, wherein the reference frame determiner is further configured to determine the reference frame from the model based on the second ultrasound frame and the second bio-signal.

The model generator may be configured to generate the model based on grouping the first ultrasound frames based on times included in the period of the first bio-signal.

The model generator may be configured to separate the acquired first bio-signal into units of one period, combine the separated one period units of the acquired first bio-signal to generate a reference bio-signal, and generate the model by mapping the generated first ultrasound frames to the reference bio-signal based on times included in the reference bio-signal.

The model generator may be configured to detect a time when each of the first bio-signal repeats similar patterns and to determine peak values of the first bio-signal, by using the acquired first bio-signals, and to separate the first bio-signal into units of one period by using the detected time and peak values.

The reference frame may be a frame that has a highest correlation with the generated second ultrasound from among the generated at least one first ultrasound frames.

The apparatus may further include a storage configured to store the generated first ultrasound frames.

According to another aspect of one or more exemplary embodiment, a method of generating an image of an object having periodic motion, by using ultrasound waves includes: detecting a first bio-signal of the object during one period of the periodic motion; generating first ultrasound frames by using first ultrasound waves transmitted to a region of interest (ROI) of the object during the one period of the periodic motion; generating a second ultrasound frame by using a second ultrasound wave transmitted to the ROI, and detecting a second bio-signal of the object during another period of the periodic motion; and selecting a reference frame from among the first ultrasound frames based on the second ultrasound frame and the second bio-signal.

The reference frame may be selected so that a position of the object in the reference frame corresponds to a position of the object in the second ultrasound frame.

### BRIEF DESCRIPTION OF THE DRAWINGS

These and/or other aspects will become apparent and more readily appreciated from the following description of exemplary embodiments, taken in conjunction with the accompanying drawings of which:

FIG. 1 is a block diagram illustrating an example of an ultrasound processing apparatus according to an exemplary embodiment;

FIG. 2 shows diagrams of an example of an operation of a model generator according to an exemplary embodiment;

FIG. 3 shows diagrams of an example of an operation of a reference frame determiner according to an exemplary embodiment;

FIG. 4 is a flowchart for describing an example of an operation of the ultrasound processing apparatus according to an exemplary embodiment;

FIG. 5 is a block diagram illustrating an example of an ultrasound system according to an exemplary embodiment; and

FIG. 6 is a flowchart for describing an example of a method of generating an image by using ultrasound waves according to an exemplary embodiment.

### DETAILED DESCRIPTION

Reference will now be made in detail to exemplary embodiments with reference to the accompanying drawings, wherein like reference numerals refer to the like elements throughout. In this regard, exemplary embodiments may have different forms and should not be construed as being limited to the descriptions set forth herein. Accordingly, the descriptions below, by referring to the figures, are merely to explain aspects of one or more exemplary embodiment. Expressions such as “at least one of,” when preceding a list of elements, modify the entire list of elements and do not modify the individual elements of the list.

The following exemplary embodiments are not limiting in scope. Thus, persons having skill in the art may infer other exemplary embodiments that fall within the scope of the claims.

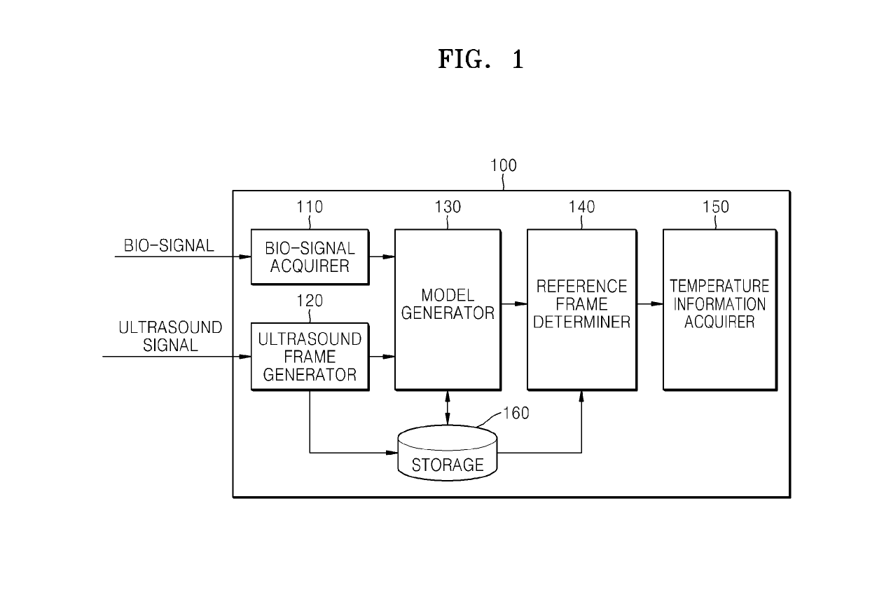

FIG. 1 is a block diagram illustrating an example of an ultrasound processing apparatus 100 according to an exemplary embodiment.

Referring to FIG. 1, the ultrasound processing apparatus 100 includes a bio-signal acquirer 110, an ultrasound frame generator 120, a model generator 130, a reference frame determiner 140, a storage 160, and a temperature information acquirer 150.

However, it can be understood by those skilled in the art that the ultrasound processing apparatus 100 may further include general-use elements in addition to the elements of FIG. 1.

Each of the bio-signal acquirer 110, ultrasound frame generator 120, model generator 130, reference frame determiner 140, storage 160, and temperature information acquirer 150 of the ultrasound processing apparatus 100 of FIG. 1 may be provided as separate devices.

The bio-signal acquirer 110, ultrasound frame generator 120, model generator 130, reference frame determiner 140, storage 160, and temperature information acquirer 150 of the ultrasound processing apparatus 100 of FIG. 1 may be implemented by using one or more processors. Each of the processors may be implemented as an array of a plurality of logic gates, or may be implemented as a combination of a processor and a memory that stores a program executable by the processor. Also, it can be understood by those skilled in the art that the bio-signal acquirer 110, ultrasound frame generator 120, model generator 130, reference frame determiner 140, storage 160, and temperature information acquirer 150 of the ultrasound processing apparatus 100 of FIG. 1 may be implemented by using other type of hardware.

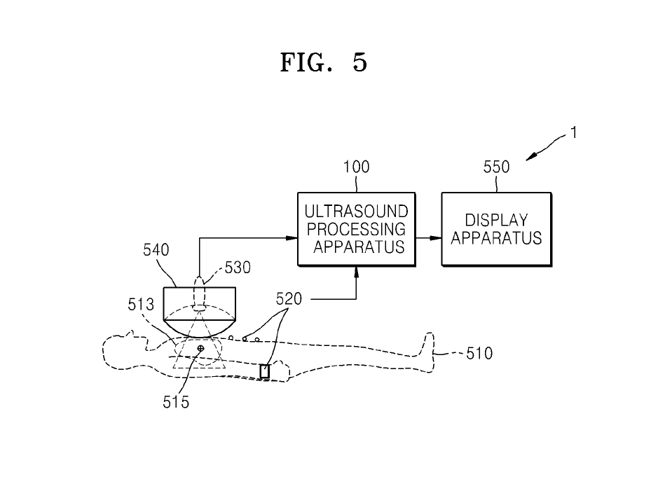

The bio-signal acquirer 110 acquires bio-signals of an object. Specifically, the bio-signal acquirer 110 receives bio-signals respectively obtained from a plurality of sensors (not shown) attached to the object (not shown) (for example, a patient) (see, for example, FIG. 5). Each of the bio-signals may correspond to a breath, a pulse, or the like of the object, but exemplary embodiments are not limited thereto. The bio-signal acquirer 110 acquires bio-signals during at least two or more periods of time. The object may have a periodic motion, and the bio-signal acquirer may detect a bio-signal during a period of periodic motion of the object. As non-limiting examples, the periodic motion may correspond to a pule or breathing.

Even when the object does not move, internal tissues of the object may slightly move. For example, when the object breathes, the shapes and positions of the internal tissues of the object may change due to inhalation and exhalation. According to an exemplary embodiment, the bio-signal acquirer 110 acquires a bio-signal when an ultrasound wave is transmitted to an ROI. Therefore, the reference frame determiner 140 may determine the most similar frame to an ultrasound frame which is currently acquired from among a plurality of previously generated ultrasound frames by using the acquired bio-signal. The most similar frame denotes a frame including the most similar information to shapes and positions of tissues shown in the currently acquired ultrasound frame.

The ultrasound frame generator 120 generates at least one ultrasound frames by using echo signals obtained from ultrasound waves which are transmitted to a preset ROI of an object. Each of the ultrasound frames includes information about an internal ROI of the object. The ROI may include an internal lesion of the object, but is not limited thereto. If the object has a periodic motion, the ultrasound waves may be transmitted during a period of the periodic motion. The ultrasound waves may be transmitted contemporaneously with the bio-signal acquirer detecting the bio-signal.

Specifically, each of a plurality of transducer elements (not shown) included in a probe (not shown) transmits an ultrasound wave to a preset ROI of an object, and receives an echo signal from the ROI. The probe (not shown) transmits the received echo signal to the ultrasound frame generator 120, which converts the received echo signal from an analog form to a digital one to generate sampling data. The ultrasound frame generator 120 performs reception beamforming on the sampling data to generate reception focusing data, and generates an ultrasound frame by using the reception focusing data. The ultrasound frame may be a radio frequency (RF) frame, but is not limited thereto.

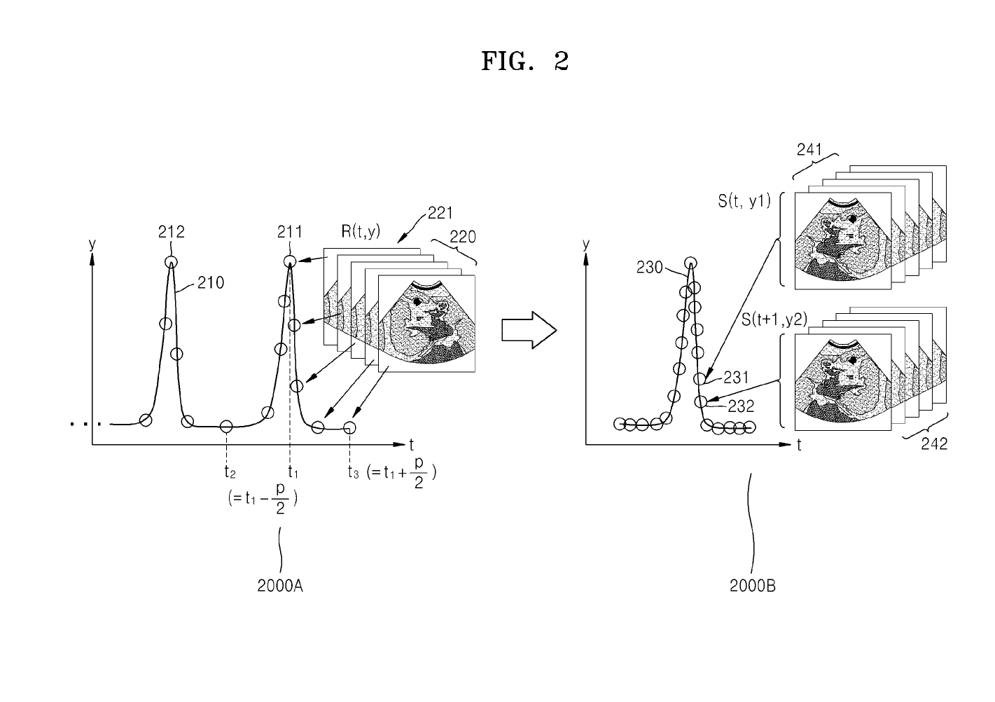

According to an exemplary embodiment, the bio-signal acquirer 110 acquires a bio-signal and the ultrasound frame generator 120 generates an ultrasound frame at the same time. In other words, a plurality of the bio-signals acquired by the bio-signal acquirer 110 and a plurality of the ultrasound frames generated at the same time by the ultrasound frame generator 120 are mapped to each other in a one-to-one relationship. This will be described in detail with reference to FIG. 2 2000A.

FIG. 2 shows diagrams of an example of an operation of a model generator according to an exemplary embodiment.

Referring to FIG. 2 2000A, a time-displacement graph shows an example of first bio-signals 210 acquired by the bio-signal acquirer 110. Also, an example of first ultrasound frames 220 generated by the ultrasound frame generator 120 is shown. Each of the first ultrasound frames 220 denotes a frame generated based on echo signals obtained from ultrasound waves transmitted at respective times when the first bio-signals 210 are acquired.

Specifically, FIG. 2 2000A shows an example in which a first bio-signal 211 acquired by the bio-signal acquirer 110 and a first ultrasound frame 221 generated by the ultrasound frame generator 120 are mapped to each other via a one-to-one relationship.

The bio-signal acquirer 110 acquires the first bio-signals 210 during at least two periods. Each of the first bio-signals 210 may correspond to a breath, a pulse, or the like of an object. Also, the ultrasound frame generator 120 generates the first ultrasound frames 220 by using echo signals obtained from ultrasound waves transmitted at respective times when the first bio-signals 210 are acquired.

In other words, the bio-signal acquirer 110 acquires the first bio-signal 211 when a probe (not shown) transmits an ultrasound wave to an ROI. The ultrasound frame generator 120 receives an echo signal obtained from the ultrasound wave transmitted from the probe (not shown) to generate the first ultrasound frame 221. Therefore, the ultrasound frame 221 generated by the ultrasound frame generator 120 may be mapped to the bio-signal 211 acquired by the bio-signal acquirer 110 via a one-to-one relationship at the same time.

Referring again to FIG. 1, the bio-signal acquirer 110 transfers information about the acquired bio-signal to the reference frame determiner 140, and the ultrasound frame generator 120 transfers the generated ultrasound frame to the reference frame determiner 140.

Generally, a degree of change of an internal tissue of an object due to a pulse, a breath, or the like of the object is generally constant or varies slightly between pulse or breath periods. In other words, a position or size of a tissue varies slightly between a time in a first period of a bio-signal and a corresponding time in a second period of the bio-signal. Therefore, the bio-signal acquirer 110 and the ultrasound frame generator 120 may respectively acquire the bio-signals and the ultrasound frames during at least two periods, and thus, the reference frame determiner 140 may select as a reference frame the most similar frame to a current status (i.e., a current change in a position or a size) of a tissue in the ROI. Although at least two periods are described, this is only an example, and exemplary embodiments may acquire bio-signals and ultrasound frames during at least three or more periods.

The model generator 130 generates a model based on one period of a bio-signal by using the first bio-signals transferred from the bio-signal acquirer 110 and the first ultrasound frames transferred from the ultrasound frame generator 120. The model denotes grouping the first ultrasound frames at each of times included in the one period of the first bio-signal. The reference frame determiner 140 determines a reference frame from the model generated by the model generator 130, based on a second generated ultrasound frame and a second acquired bio-signal. As a non-limiting example, the reference frame may be selected so that a position of the object in the reference frame corresponds to a position of the object in the currently acquired ultrasound frame. Hereinafter, an example in which the model generator 130 generates the model will be described with reference to FIG. 2.

Referring to FIG. 2 2000B, an example in which the model generator 130 groups a plurality of first ultrasound frames 241 and 242 at each of times 231 and 232 when first bio-signals included in one period 230 are respectively acquired is shown. A detailed method in which the model generator 130 generates the model is as follows.

First, the model generator 130 separates the first bio-signals 210, transferred from the bio-signal acquirer 110, in units of one period. A method, in which the model generator 130 separates the first bio-signals 210 in units of one period, will be described below with reference to FIG. 2 2000A.

The model generator 130 performs a fast Fourier transform (FFT) on the first bio-signals 210 to detect a period p. For example, the model generator 130 analyzes the FFT result to detect the period p that denotes a time when a bio-signal repeats similar patterns. Furthermore, the model generator 130 detects peak values from the first bio-signals 210, respectively. However, these are just examples and the period p may be detected using other means.

The model generator 130 separates the first bio-signals 210 in units of one period by using the detected period p and peak values. For example, the model generator 130 finds a peak value at a time close to a time that elapses by the period p from one peak value 212 of the peak values. That is, the model generator 130 finds another peak value 211 at a time that elapses by “p±Δt” from the one peak value 212 of the peak values. Here, Δt denotes a certain time shorter than the period p. Subsequently, the model generator 130 calculates a time t2 (=t1−p/2) and a time t3 (=t1+p/2) with respect to a time t1 when the found peak value 211 is found. Then, the model generator 130 separates bio-signals, which are respectively acquired between the times t2 and t3, from the first bio-signals 210. The model generator 130 may repeat the above-described operations to separate the bio-signals 210 in units of one period.

Referring again to FIG. 2 2000B, the model generator 130 combines the separated bio-signals of one period unit to generate a reference bio-signal 230. For example, the model generator 130 may align the separated bio-signals in units of one period to generate the reference bio-signal 230. Here, the reference frame determiner 140 may perform Gaussian fitting by using upper 50% or more values of displacement values of the bio-signals separated in units of one period to align the bio-signals separated in units of one period, but is not limited thereto.

The model generator 130 maps the first ultrasound frames 241 and 242 to the reference bio-signal 230 on the basis of the times 231 and 232 included in the reference bio-signal 230, thereby generating a model. For example, the model generator 130 may group the first ultrasound frames 241 and 242 on the basis of echo signals obtained from ultrasound waves that are irradiated at respective times corresponding to the times 231 and 232 included in the reference bio-signal 230. As described above, the bio-signal acquirer 110 and the ultrasound frame generator 120 respectively acquire the first bio-signals and the first ultrasound frames during at least two periods, and thus, at least two ultrasound frames 241 and 242 are grouped at each of the times 231 and 232 included in the reference first bio-signal 230.

Referring again to FIG. 1, the above-described bio-signal acquirer 110, ultrasound frame generator 120, and model generator 130 may operate before a medical procedure of an object. In other words, before the medical procedure of the object, the model generator 130 may generate the model by using the first bio-signals acquired by the bio-signal acquirer 110 and the first ultrasound frames generated by the ultrasound frame generator 120. The below-described reference frame determiner 140 may determine the reference frame by using second bio-signal-acquired by the bio-signal acquirer 110 and second ultrasound frame generated by the ultrasound frame generator 120 while the medical procedure of the object is being performed. The second bio-signal may be of the same type as the first bio-signal. For instance, if the first bio-signal corresponds to a breath, the second bio-signal corresponds to a breath. The primary difference between the first and second bio-signals may then be the time in which the bio-signal is acquired.

The storage 160 may store the first bio-signals acquired by the bio-signal acquirer 110, the first ultrasound frames generated by the ultrasound frame generator 120, and the first model generated by the model generator 130.

The reference frame determiner 140 determines the reference frame from the first ultrasound frames on the basis of the generated second ultrasound frame and the acquired second bio-signal. Here, the reference frame denotes a frame having the highest correlation with the generated second ultrasound frames among the generated second ultrasound frames.

Specifically, the reference frame determiner 140 determines the reference frame from the model generated by the model generator 130, on the basis of the second bio-signal acquired by the bio-signal acquirer 110 and the second ultrasound frame generated by the ultrasound frame generator 120. Here, while the medical procedure of the object is being performed, the bio-signal acquirer 110 may acquire the second bio-signal, and the ultrasound frame generator 120 may generate the second ultrasound frame.

An operation to compare all of the first ultrasound frames generated before the medical procedure of the object and the second ultrasound frame generated during the medical procedure is needed for finding the most similar frame to the second ultrasound frame generated during the medical procedure from among the first ultrasound frames generated before the medical procedure. Therefore, as a large number of comparisons is performed in the comparison operation, the comparison operation takes a long time.

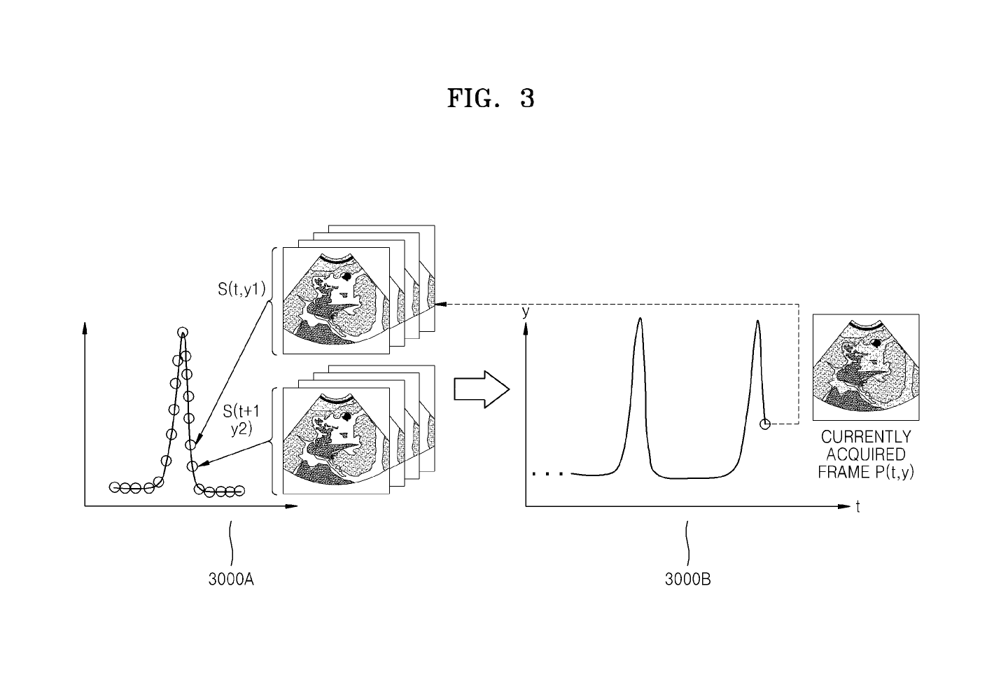

The reference frame determiner 140 according to an exemplary embodiment determines the reference frame by using the generated model. In other words, the model generated by the model generator 130 includes a plurality of sets including first ultrasound frames that are grouped by time intervals included in one period of a bio-signal, and thus, the reference frame determiner 140 selects one set from among the plurality of sets included in the model, and determines the reference frame from among the first ultrasound frames included in the selected set. Therefore, an operation of comparing the generated second frame with all of the generated first ultrasound frames is not needed, thereby reducing the number of operations and a computation time for determining the reference frame. Rather, a reference frame may be selected by comparing the generated second frame with only the first ultrasound frames included in the selected set. Hereinafter, an example of an operation of the reference frame determiner 140 will be described with reference to FIG. 3.

FIG. 3 includes diagrams showing an example of an operation of the reference frame determiner 140 according to an exemplary embodiment.

Referring to FIG. 3 3000A, an example of the model generated by the model generator 130 is shown. In other words, the drawing shown in FIG. 3 3000A corresponds to the drawing shown in FIG. 2 2000B. FIG. 3 3000A shows a plurality of sets (S(t, y1), S(t+1, y2)) including first ultrasound frames that are grouped at two times t and t+1 of a plurality of times in one period of a first bio-signal.

Referring to FIG. 3 3000B, the reference frame determiner 140 selects one set from among a plurality of sets including first ultrasound frames included in a group. The set selected by the reference frame determiner 140 denotes a set (S(t, y1)) corresponding to a time when a first bio-signal is acquired by the bio-signal acquirer 110.

Specifically, the reference frame determiner 140 detects an acquisition time t and a displacement y from a second bio-signal acquired by the bio-signal acquirer 110. The second bio-signal acquired by the bio-signal acquirer 110 denotes a bio-signal acquired when a probe (not shown) transmits a second ultrasound wave on an ROI, similar to the first bio-signal. If the object has a periodic motion, the second ultrasound wave may be transmitted and the second bio-signal may be detected during a period of the periodic motion different from the period during which the first bio-signal was detected.

The reference frame determiner 140 selects the set (S(t, y1)) from among a plurality of first ultrasound frame sets included in the model.

The reference frame determiner 140 compares a second ultrasound frame (P(t, y)) generated by the ultrasound frame generator 120 and the first ultrasound frames included in the selected set (S(t, y1)), and determines the most similar frame to the generated second ultrasound frame (P(t, y)) from among the first ultrasound frames included in the selected set (S(t, y1)). The second ultrasound frame (P(t, y)) generated by the ultrasound frame generator 120 denotes an ultrasound frame generated by using an echo signal obtained from an ultrasound wave which is re-transmitted to the ROI by the probe (not shown), similar to the first ultrasound frames.

The reference frame determiner 140 may compare the generated second ultrasound frame (P(t, y)) and the first ultrasound frames included in the selected set (S(t, y1)) by using the following Equation (1):

$$r = \frac{{Σ}_{m} {Σ}_{n} ( {A}_{mn} - \overline{A} ) ( {B}_{mn} - \overline{B} )}{\sqrt{( {Σ}_{m} {{Σ}_{n} ( {A}_{mn} - \overline{A} )}^{2} ) ( {( {B}_{mn} - \overline{B} )}^{2} )}} \qquad ( 1 )$$

where r denotes a correlation between the generated second ultrasound frame (P(t, y)) and the first ultrasound frames included in the selected set (S(t, y1)). A denotes the selected set (S(t, y1)), m denotes number of first ultrasound frames included in the selected set (S(t, y1)), and Ā denotes an average of the first ultrasound frames included in the selected set (S(t, y1)). B denotes the generated second ultrasound frame (P(t, y)), n denotes the number (for example, one) of generated second ultrasound frames (P(t, y)), and B̅ denotes an average (for example, zero) of the generated second ultrasound frames (P(t, y)).

The reference frame determiner 140 determines as the reference frame a first ultrasound frame having the highest correlation r with the generated second ultrasound frame (P(t, y)) from among the first ultrasound frames included in the selected set (S(t, y1)) by using Equation (1).

As described above, the reference frame determiner 140 determines the reference frame from among a plurality of first ultrasound frames which are previously acquired by using bio-signals of an object, thereby quickly and accurately determining the reference frame.

Referring again to FIG. 1, the reference frame determiner 140 transfers the determined reference frame and the generated second ultrasound frame to the temperature information acquirer 150.

The temperature information acquirer 150 acquires information indicating a temperature change of the ROI by using the generated second ultrasound frame and the reference frame.

For example, the temperature information acquirer 150 compares a radio frequency (RF) signal included in the reference frame and an RF signal included in the generated second ultrasound frame to detect an amplitude-changed portion of the RF signal included in the generated second ultrasound frame. The temperature information acquirer 150 acquires information (for example, a change value of a temperature) indicating the temperature change of the ROI corresponding to a degree of detected amplitude change by using a mapping table stored in the storage 160.

The mapping table may include predetermined amplitude change values of a plurality of echo signals and a plurality of temperature change values which are respectively mapped to the amplitude change values. A temperature change value mapped to one amplitude change value in the mapping table denotes a temperature change of an ROI predicted from the amplitude change value.

Moreover, the temperature information acquirer 150 may generate a temperature map (corresponding to a relative temperature change of a part of interest) for a current frame by using the acquired information.

As described above, the ultrasound processing apparatus 100 previously acquires first ultrasound frames indicating an ROI during a certain period of a bio-signal of an object before monitoring a temperature change of the ROI, and determines a reference frame from among the previously acquired first ultrasound frames, thereby accurately monitoring the temperature change of the ROI.

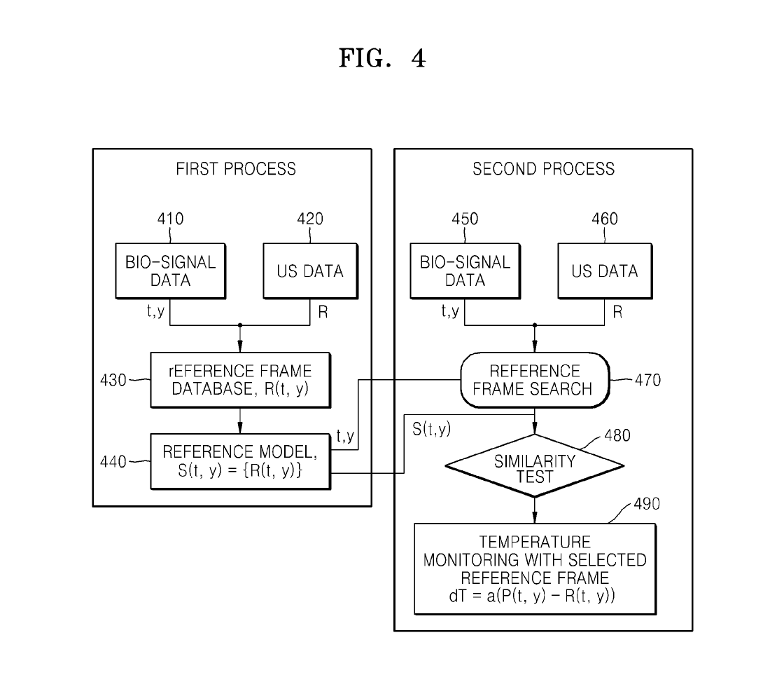

FIG. 4 is a flowchart for describing an example of an operation of the ultrasound processing apparatus according to an exemplary embodiment.

Referring to FIG. 4, an example in which the elements included in the ultrasound processing apparatus 100 operates with time is illustrated. In FIG. 4, a first process denotes a process that is performed before a medical procedure for diagnosing or treating an object, and a second process denotes a process that is performed during the medical procedure for diagnosing or treating the object. The medical procedure may denote treating a lesion in an ROI by focusing a high intensity focused ultrasound (HIFU) wave on the lesion, but exemplary embodiments are not limited thereto.

The descriptions with reference to FIGS. 1 to 3 may also apply to the flowchart of FIG. 4.

In operation 410, the bio-signal acquirer 110 acquires first bio-signals during at least two periods. Each of the first bio-signals may correspond to a breath or a pulse of the object, but exemplary embodiments are not limited thereto.

In operation 420, the ultrasound frame generator 120 generates a first ultrasound frame by using an echo signal obtained from an ultrasound wave which is transmitted to an ROI of the object by a probe (not shown) when a corresponding first bio-signal is acquired. A plurality of the first ultrasound frames generated by the ultrasound frame generator 120 may be mapped to the first bio-signals acquired by the bio-signal acquirer 110 via a one-to-one relationship.

In operation 430, the ultrasound frame generator 120 stores the generated first ultrasound frame in the storage 160.

In operation 440, the model generator 130 generates a model by using the first bio-signals and the plurality of first ultrasound frames. The model denotes grouping the first ultrasound frames at each of times included in one period of each of the first bio-signals.

In operation 450, the bio-signal acquirer 110 acquires a second bio-signal during a medical procedure. The second bio-signal is of the same type as the first bio-signal. For instance, if the first bio-signal corresponds to a breath, the second bio-signal corresponds to a breath. The primary difference between the first and second bio-signals may then be the time in which the bio-signal is acquired.

In operation 460, the ultrasound frame generator 120 generates a second ultrasound frame by using an echo signal obtained from an ultrasound wave which is re-transmitted to the ROI by the probe (not shown) when the second bio-signal is acquired.

In operation 470, the reference frame determiner 140 selects a set corresponding to the acquired second bio-signal from among a plurality of sets including a plurality of first ultrasound frames included in the model. At this time, the reference frame determiner 140 detects an acquisition time and a displacement from the acquired second bio-signal, and selects one set from among the plurality of sets included in the model by using the detected acquisition time and displacement.

In operation 480, the reference frame determiner 140 determines a reference frame that is most similar to a generated second ultrasound frame (i.e., an ultrasound frame generated during the medical procedure) among a plurality of first ultrasound frames included in the selected set. The reference frame determiner 140 may determine the reference frame by using Equation (1).

In operation 490, the temperature information acquirer 150 acquires information about a temperature change of the ROI by using the generated second ultrasound frame and the reference frame. Also, the temperature information acquirer 150 may generate a temperature map indicating the temperature change of the ROI.

FIG. 5 is a block diagram illustrating an example of an ultrasound system 1 according to an exemplary embodiment.

Referring to FIG. 5, the ultrasound system 1 may include a probe 530, an HIFU irradiating apparatus 540, and a display apparatus 550, in addition to the ultrasound processing apparatus 100. Also, the ultrasound system 1 may further include a sensor 520 that acquires a bio-signal.

An operation performed by the ultrasound processing apparatus 100 is as described above with reference to FIGS. 1 to 4, and thus, its detailed description is not provided below.

The probe 530 transmits an ultrasound wave to an ROI 513 in an object 510, and receives an echo signal therefrom. Furthermore, the probe 530 transfers the received echo signal to the ultrasound processing apparatus 100 (specifically, the ultrasound frame generator 120).

The sensor 520 acquires a bio-signal (for example, corresponding to a pulse or a breath) of the object 510. The sensor 520 transfers the acquired bio-signal to the ultrasound processing apparatus 100 (specifically, the bio-signal acquirer 110). The sensor 520 may be a plurality of sensors that acquire one or more bio-signals. As a non-limiting examples, a plurality of sensors may be used to acquire a breath and a pulse. The sensor 520 may transfer a plurality of bio-signals to the ultrasound processing apparatus 100.

The HIFU generating apparatus 540 focuses an HIFU wave on a lesion 515 in the ROI 513. The ultrasound system 1 according to an exemplary embodiment may optionally include the HIFU generating apparatus 540.

The display apparatus 550 may receive an image from the ultrasound processing apparatus 100, and display the image on a screen. The image may be a B-mode image of the ROI generated by the ultrasound frame generator 120 or a temperature map image of the ROI generated by the temperature information acquirer 150.

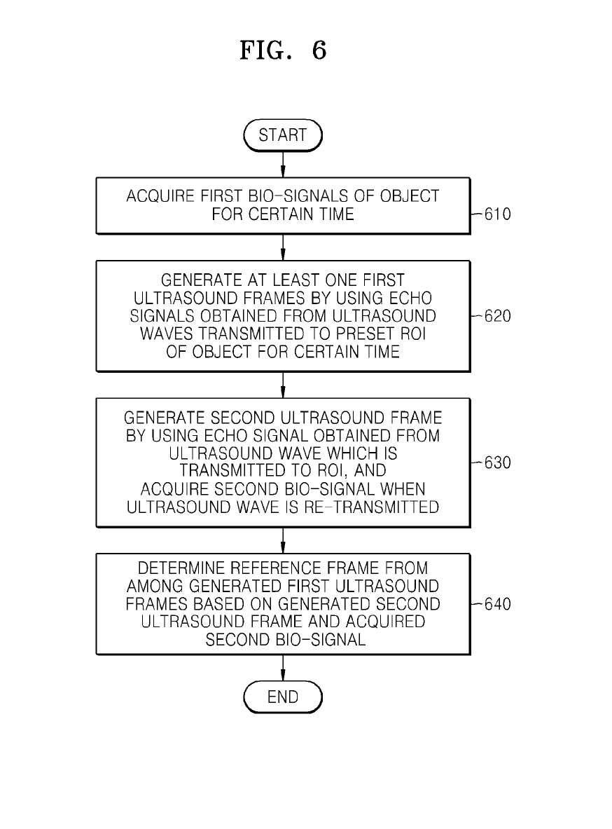

FIG. 6 is a flowchart for describing an example of a method of generating an image by using ultrasound waves according to an exemplary embodiment.

Referring to FIG. 6, the method of generating an image by using ultrasound waves includes a plurality of operations that are performed by the ultrasound processing apparatus 100 or the ultrasound system 1 illustrated in FIGS. 1 and 5. Thus, although not described below, the descriptions of the ultrasound processing apparatus 100 or the ultrasound system 1 illustrated in FIGS. 1 and 5 are also valid for the method of generating an image by using ultrasound waves in FIG. 6.

In operation 610, the bio-signal acquirer 110 acquires first bio-signals of an object for a certain time. The bio-signal acquirer 110 acquires first bio-signals during at least two periods of the first bio-signals.

In operation 620, the ultrasound frame generator 120 generates at least one first ultrasound frames by using echo signals obtained from ultrasound waves which are transmitted to a preset ROI of the object for a certain time. That is, the ultrasound frame generator 120 generates during the time period when the first bio-signals are acquired the at least one first ultrasound frames by using the echo signals obtained from the ultrasound waves transmitted to the ROI.

In operation 630, the ultrasound frame generator 120 generates a second ultrasound frame by using an echo signal obtained from an ultrasound wave which is transmitted to the ROI, and the bio-signal acquirer 110 acquires a second bio-signal at a time when the ultrasound wave is transmitted. The second bio-signal is of the same type as the first bio-signal. For instance, if the first bio-signal corresponds to a breath, the second bio-signal corresponds to a breath. The primary difference between the first and second bio-signals may then be the time in which the bio-signal is acquired.

In operation 640, based on the generated second ultrasound frame and the acquired second bio-signal, the reference frame determiner 140 determines a reference frame from among the generated first ultrasound frames. At this time, the reference frame determiner 140 may select one set from among a plurality of first ultrasound frame sets included in a model generated by the model generator 130, and determine the reference frame from among a plurality of first ultrasound frames included in the selected set.

As described above, according to the one or more of the above exemplary embodiments, first ultrasound frames indicating an ROI are previously acquired during a certain period of a first bio-signal of an object before monitoring a temperature change of the ROI, and a reference frame is determined from among previously acquired first ultrasound frames, thereby accurately monitoring the temperature change of the ROI.

Moreover, the reference frame is determined from among the previously acquired first ultrasound frames by using the first and second bio-signals of the object, and thus, the temperature change of the ROI is quickly monitored.

The above-described method may be embodied as computer programs and may be implemented in general-use digital computers that execute the programs via a non-transitory computer-readable recording medium. Data structure used in the above-described method may be recorded in a computer-readable recording medium by using various methods. Non-limiting examples of the non-transitory computer-readable recording medium include magnetic storage media (e.g., ROM, RAM, USB, floppy disks, hard disks, etc.) and storage media such as optical recording media (e.g., CD-ROMs, or DVDs) and PC interfaces (e.g., PCI, PCI-express, Wi-Fi, etc.).

It should be understood that the exemplary embodiments described herein should be considered in a descriptive sense only and not for purposes of limitation. Descriptions of features or aspects within each embodiment should typically be considered as available for other similar features or aspects in other exemplary embodiments.

What is claimed is:

1. A method of generating an image by using ultrasound waves, the method comprising:
acquiring a first bio-signal of an object;
generating at least one first ultrasound frame by using at least one echo signal obtained from at least one first ultrasound wave transmitted to a preset region of interest (ROI) of the object;
generating a second ultrasound frame by using an echo signal obtained from a second ultrasound wave transmitted to the preset ROI, and acquiring a second bio-signal of the object at a time when the second ultrasound wave is transmitted; and
determining a reference frame from among the generated at least one first ultrasound frame based on the generated second ultrasound frame and the acquired second bio-signal.

2. The method of claim 1, further comprising acquiring information about a temperature change of the preset ROI by using the generated second ultrasound frame and the determined reference frame.

3. The method of claim 1, further comprising generating a model based on a period of the first bio-signal by using the acquired first bio-signal and the generated at least one first ultrasound frames,
wherein the determining of the reference frame comprises determining the reference frame from the model based on the generated second ultrasound frame and the acquired second bio-signal.

4. The method of claim 3, wherein the model is based on grouping the at least one first ultrasound frame based on time intervals included in the period of the first bio-signal.

5. The method of claim 3, wherein the generating of the model comprises:
separating the acquired first bio-signal into units of one period;
combining the separated one period units of the first bio-signal to generate a reference bio-signal; and
generating the model by mapping the at least one first ultrasound frame to the reference bio-signal based on time intervals included in the reference bio-signal.

6. The method of claim 5, wherein the separating comprises:
detecting a time when the first bio-signal repeats similar patterns and determining peak value of the bio-signal, by using the acquired bio-signal; and
separating the bio-signal into units of one period by using the detected time and the peak values.

7. The method of claim 1, wherein the reference frame is a frame that has a highest correlation with the generated second ultrasound from among the generated at least one first ultrasound frames.

8. The method of claim 1, further comprising storing the generated at least one first ultrasound frames.

9. A non-transitory computer-readable storage medium storing a computer program for executing the method of claim 1 in a computer.

10. An apparatus configured to generate an image by using ultrasound waves, the apparatus comprising:
a bio-signal acquirer configured to acquire a first bio-signal of an object;
an ultrasound frame generator configured to generate a plurality of first ultrasound frames by using echo signals obtained from first ultrasound waves transmitted to a preset region of interest (ROI) of the object; and
a reference frame determiner configured to determine a reference frame from among the plurality of first ultrasound frames based on a second ultrasound frame and a second bio-signal,
wherein, the ultrasound frame generator is further configured to generate the second ultrasound frame by using an echo signal obtained from a second ultrasound wave transmitted the preset ROI, and
the bio-signal acquirer is further configured to acquire the second bio-signal at a time when the second ultrasound wave is transmitted to the preset ROI.

11. The apparatus of claim 10, further comprising a temperature information acquirer configured to acquire information about a temperature change of the preset ROI by using the second ultrasound frame and the reference frame.

12. The apparatus of claim 10, further comprising a model generator configured to generate a model based on a period of the first bio-signal by using the acquired first bio-signal and the generated first ultrasound frames,
wherein the reference frame determiner is further configured to determine the reference frame from the model based on the second ultrasound frame and the second bio-signal.

13. The apparatus of claim 12, wherein the model generator is further configured to generate the model based on grouping the first ultrasound frames based on times included in the period of the first bio-signal.

14. The apparatus of claim 12, wherein the model generator is further configured to separate the acquired first bio-signal into units of one period, combine the separated one period units of the acquired first bio-signal to generate a reference bio-signal, and generate the model by mapping the generated first ultrasound frames to the reference bio-signal based on times included in the reference bio-signal.

15. The apparatus of claim 14, wherein the model generator is further configured to detect a time when each of the first bio-signal repeats similar patterns and to determine peak values of the first bio-signal, by using the acquired first bio-signals, and to separate the first bio-signal into units of one period by using the detected time and peak values.

16. The apparatus of claim 10, wherein the reference frame is a frame that has a highest correlation with the generated second ultrasound frame from among the generated first ultrasound frames.

17. The apparatus of claim 10, further comprising a storage configured to store the generated first ultrasound frames.

18. A method of generating an image of an object having a periodic motion, by using ultrasound waves, the method comprising:
detecting a first bio-signal of the object during one period of the periodic motion;
generating first ultrasound frames by using first ultrasound waves transmitted to a region of interest (ROI) of the object during the one period of the periodic motion;
generating a second ultrasound frame by using a second ultrasound wave transmitted to the ROI, and detecting a second bio-signal of the object during another period of the periodic motion; and
selecting a reference frame from among the first ultrasound frames based on the second ultrasound frame and the second bio-signal.

19. The method of claim 18, wherein reference frame is selected so that a position of the object in the reference frame corresponds to a position of the object in the second ultrasound frame.
